(19) United States
(12) Patent Application Publication
(10) Pub. No.: US 2001/0055801 A1
(43) Pub. Date: Dec. 27, 2001

(54) Liquid arrays

(76) Inventors: Shiping Chen, Rockville, MD (US); Yuling Luo, Castro Valley, CA (US); Anthony C. Chen, Sunnyvale, CA (US)

Correspondence Address: MORRISON & FOERSTER LLP, 755 PAGE MILL RD, PALO ALTO, CA, 94304-1018, US

(21) Appl. No.: 09/791,411

(22) Filed: Feb. 22, 2001

Related U.S. Application Data
(60) Provisional application No. 60/183,737, filed on Feb. 22, 2000.
(60) Provisional application No. 60/188,872, filed on Mar. 13, 2000.
(60) Provisional application No. 60/216,265, filed on Jul. 6, 2000.
(60) Provisional application No. 60/220,085, filed on Jul. 21, 2000.
(60) Provisional application No. 60/244,711, filed on Oct. 30, 2000.
(60) Provisional application No. 60/244,413, filed on Oct. 30, 2000.

(51) Int. Cl. C12M 1/34; C12Q 1/68; G01N 33/543

(52) U.S. Cl. 435/287.2; 435/6; 436/518

(57) ABSTRACT

A capillary bundle having reaction wells in one end of the capillaries is disclosed. The reaction wells serve as sites for hybridization, compound reaction, and drug identification for instance. The capillaries may be light-conducting capillaries. Also disclosed are various methods of identifying a target compound in a liquid carrier using this capillary bundle as well as methods of fabricating the bundle.

This invention claims the benefit of priority to U.S. Provisional Application Nos: 60/183,737, filed on Feb. 22, 2000; 60/188,872, filed on Mar. 13, 2000; 60/216,265, filed on Jul. 6, 2000; 60/220,085, filed on Jul. 21, 2000; 60/244,711, filed on Oct. 30, 2000; 60/244,413, filed on Oct. 30, 2000; U.S. Provisional Application Docket No. 473533000600, titled METHOD AND APPARATUS BASED ON BUNDLED CAPILLARIES FOR HIGH THROUGHPUT SCREENING by Jianming Xiao et al., filed on Feb. 16, 2001; PCT Application Docket No. 473532000240, titled MICROARRAY FABRICATION TECHNIQUES AND APPARATUS by inventors Shiping Chen, Yuling Luo, and Anthony C. Chen; and PCT Application Docket No. 473532000270, titled MICROARRAY FABRICATION TECHNIQUES AND APPARATUS by inventors Shiping Chen, Yuling Luo, and Anthony C. Chen, the latter two having been filed on even date herewith. All of the above applications are incorporated by reference herein in their entireties as if fully set forth below.

### BACKGROUND

Various DNA chip technologies have been developed to allow the analysis of gene expression in highly parallel fashion. The expression of thousands of genes can be analyzed at one time and this has been tremendously useful in the identification of genes involved in disease processes. Although the expression of a gene in a given cell in general correlates well with its protein expression, it is not always the case. In many instances, protein expression is subject to translational control, which determines if and when predicted gene products are translated. In addition, protein expression is subject to post-translational modification such as phosphorylation. In those instances, the level and activity of proteins within the cells could not be accurately predicted from their nucleic acid sequence or their gene expression pattern. Thus there is a need to study the entire complement of proteins and their expression in normal and disease states.

The current DNA chip technology is difficult to apply to protein arrays because proteins are much more fragile than DNA. Nucleic acid is very robust in nature. It can stand up to heat, can be dried and re-hydrated repeatedly, and can be attached to solid surfaces without loss of activity. In contrast, proteins become denatured and lose their activity with heat, drying, or interaction with non-compatible surface materials. Maintaining protein activity at solid-liquid interface requires different attachment strategies than those for nucleic acids. Consequently, there is a need to develop a solution based protein array system.

### DESCRIPTION OF THE INVENTION

Array Configurations

The liquid arrays in this invention comprise a large number of through holes grouped together in orderly or random fashion. Probes in liquid form are stored inside different holes. The inner diameter of the holes can range from 5 mm to less than 1 μm and average pitch of the hole array can range from 10 mm to less than 2 μm. The length of the through hole can be anywhere between 50 μm to hundreds of meters depending on the array configuration and application. The number of through holes in the array can range from 10 to 10 million.

The probes can be anything that is fit to be stored in solution and transported by through holes, including, without limitation, deoxyribonucleic acids (DNA), ribonucleic acids (RNA), sythetic oligonucleotides, antibodies, proteins, peptides, lectins, modified polysaccharides, synthetic composite macromolecules, functionalized nanostructures, synthetic polymers, modified/blocked nucleotides/nucleosides, modified/blocked amino acides, fluorophores, chromophores, ligands, chelates, haptens and drug compounds. Preferably, the probes are polypeptides.

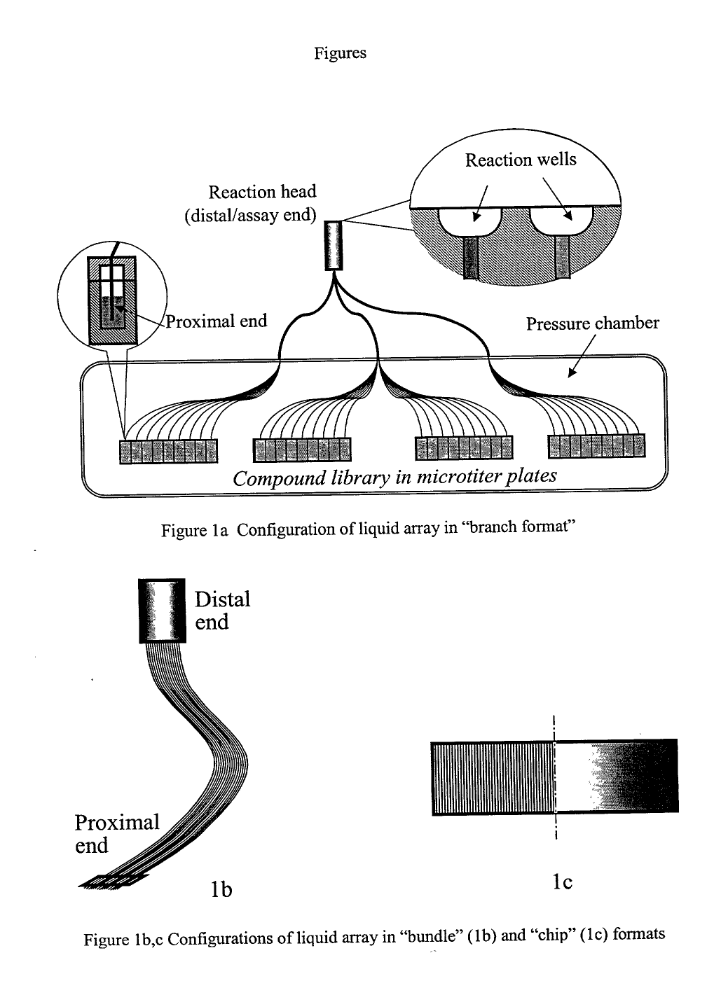

The liquid protein array of this invention has a variety of formats. In the first configuration, referred as “branch format”, as shown in FIG. 1a, through holes are formed inside individual capillaries. The length of the capillary can range from about 0.5 m to tens of meters and the outer diameter of the capillary can range from about 2 mm to about 10 m. For each capillary, the proximal end is inserted into a liquid reservoir while the distal end is bundled together with that of many other capillaries to form a solidified piece. The liquid reservoir can take the form of a well in a standard microtiter plate.

The second configuration is referred as “bundle format”, as shown in FIG. 1b. The through holes are also formed in individual capillaries with outer diameters in about 2 mm to about 10 μm range but a large number of capillaries are bundled along the entire length, either loosely or solidified. The diameter of the cavity in the capillary is small enough and inner surface of the cavity is sufficiently hydrophobic that liquid probes are retained within the cavity by capillary force. The length of bundle can range from about 0.1 m to hundreds of meters.

In the third configuration, referred as “chip format”, as shown in FIG. 1c, all through holes are formed in a solid piece, which takes a chip shape having an up and a bottom surface where through holes exit. Similar to the previous format, the diameters of the holes are small enough and inner surfaces of the holes are sufficiently hydrophobic that liquid probes are retained within the cavity by capillary force. The thickness of the chip, hence the length of the through holes, can range from about 50 μm to several tens of centimeters.

In these configurations, a microscopic reaction well is fabricated at the distal (bundled and solidified) end of each hole, as shown in FIG. 1a. This end of the liquid array is also referred to as “assay end”. The diameter of the microwell is much larger than that of the through hole. It is also possible that several holes share a well at the assay end, as shown in FIG. 2b. In the bundle and chip format, a fluid reservoir can be fabricated at the proximal end by enlarging the inner diameter of the through hole so that more liquid probes can be held within each hole, as shown in FIG. 2.

Liquid Protein Probes

The liquid protein probes are made by attaching protein molecules to microscopic ferromagnetic beads with dimension significantly smaller that the diameter of the through holes in the array. These beads are further suspended in a suitable buffer fluid. In another embodiment, the proteins are dissolved in solution phase.

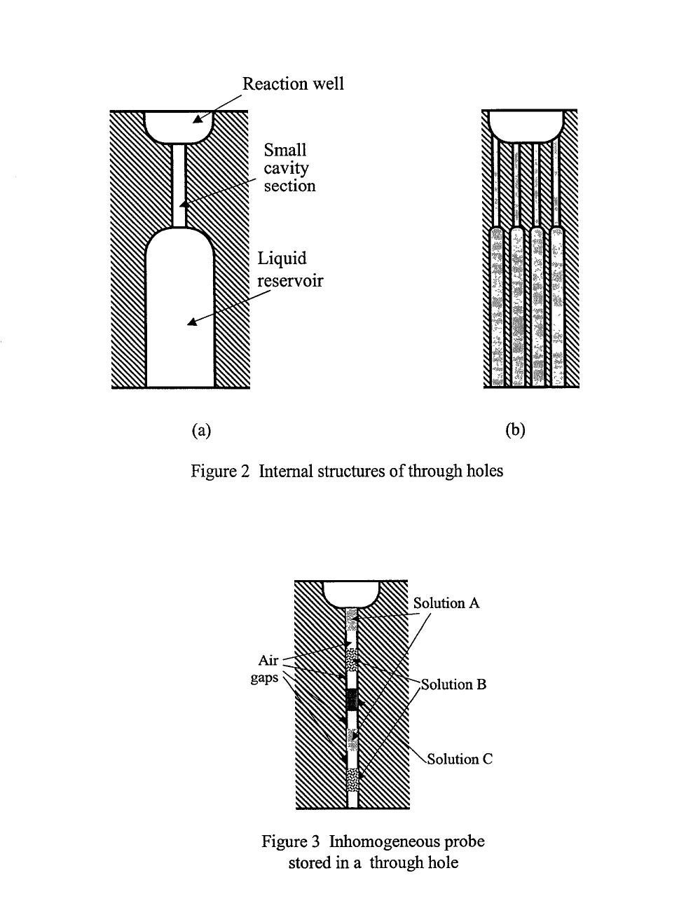

Within each through hole, the liquid probe may be either homogeneous or heterogeneous. In the later case, the probe may comprise different solutions or the same solution of different concentrations. These difference elements of the probe are distributed in different sections along the through hole and may be separated by an air gap, as illustrated in FIG. 3.

Use

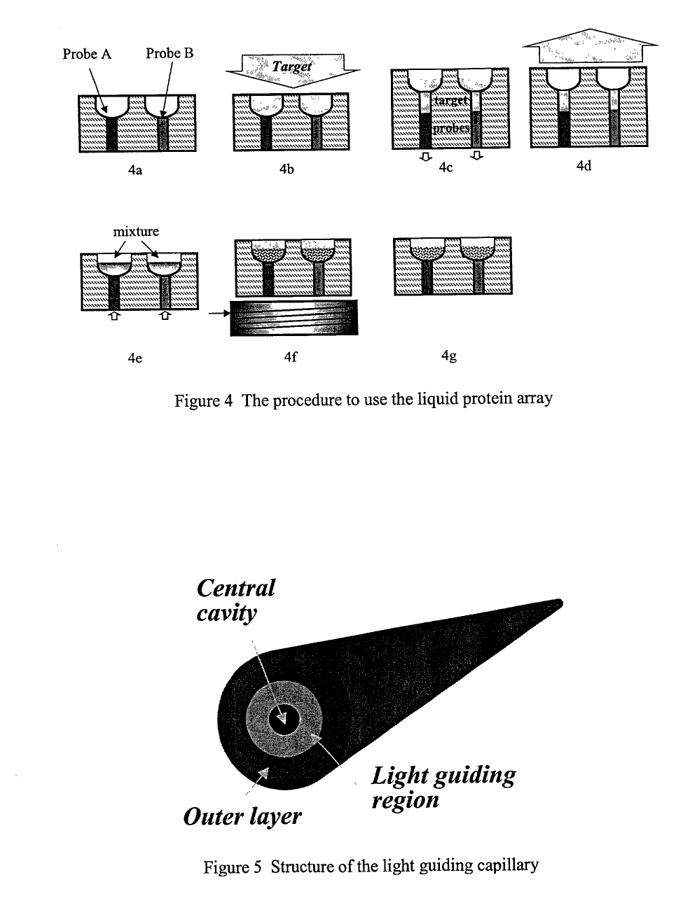

When the liquid protein arrays in this invention are used in protein analysis experiments, the proximal (or non assay) end of the array is placed inside a pressure chamber. As illustrated in FIG. 4, first, a positive pressure is applied in the pressure chamber which drives the probes near the bottom of the microwells at the assay end of the array (FIG. 4a). Then the target protein in liquid form is applied to the assay end of the array. The target is sufficient to universally fill all the microwells in the array (4b). Third, a controlled level of vacuum is applied to the pressure chamber to draw a proportion of the target liquid in the microwell into the through hole that is linked to the well from the bottom (4c). Fourth, a suitable vacuum is applied at the top (or assay end) of the array. The suction force is precisely controlled in such a way that it is large enough to suck the remaining target liquid in the wells but is too small to overcome the capillary force generated in the small through holes. As a result, the target liquid that has been drawn into through holes will remain in tact (4d). Fifth, a positive pressure is applied to the proximal end to push the target and a certain amount of the probe into the microwell and mixing and incubating them in the well (4e). Sixth, a magnetic field is generated that pulls the ferromagnetic beads with the probe attached towards the walls of each well (4f). Seventh, the assay side of the array is washed to remove unbound proteins in the target. Then the result of binding or hybridization can be imaged ultilizing microarray readout technologies currently available on the market (4g). Finally, the array can be demagnetized, the solution mixture in the well removed. After washing, the array will be ready for the next experiment (4a).

In a specific example, the through holes are 20 μm in diameter with a pitch of 100 μm across the array. The reaction wells are 80 μm in diameter and 80 μm deep, providing a 0.4 nl volume. A liquid array comprising 100,000 holes is about 30 mm in diameter. The volume of each probe used in one experiment is less than 0.4 nl. The volume of target liquid used in one experiment is less than 40 μl.

As mentioned before, each through hole in the array may contain more than a single homogenous solution, separated by a small air gap. In this case, a sequence of solutions can be pumped into the microwell for the reaction. This enables the liquid protein array to perform much more complex assays.

The branch format is suitable for continuous and repeated use for a long period of time. The bundle format can be used repeatedly for a large number of times with the length of the capillaries bundle determining the number of repeated usage. The chip format is designed for a single or a small number of usages. Assuming 20 μm in diameter, a through hole of 10 mm in length can hold 3 nl of liquid probe, which is sufficient for approximately 6 experiments. As described above, it is possible to build a fluid reservoir at the proximal end of each through hole by enlarging the inner diameter of the hole. Assuming the through hole in the above case has a 6 mm section with an enlarged inner diameter of 80 μm at the proximal end, as shown in FIG. 2, the probe volume held in the through hole would be increased to 30 nl, sufficient for about 60 experiments.

The protein probe can be dissolved in solution and not attached to beads. In addition, the inner surface of the microwell can be coated with an agent such as an antibody or avidin/streptavidin that binds or has a high affinity to the protein molecule. Such treated surface can immobilize the protein probes once they get inside the wells. Thereafter, washing can be conducted in the wells to remove nonbinding molecules from the wells.

Fabrication

Branch Format

The liquid array in branch format is assembled from individual capillaries. A capillary can be made of any suitable materials including silica, glass, plastic, polymer, metal and ceramics using standard extrusion or drawing process. In a preferred embodiment, the capillary is made of silica with a Germanium doped region built around the central cavity, as shown in FIG. 5. Such a capillary can be fabricated using Modified Chemical Vapor Deposition (MCVD) process widely used in optical fiber manufacturing. This Germanium doped region has two major utilities. First, the Germanium doping increases the optical refractive index of the silica, thus creating a waveguide through the Germanium doped region, which is capable of guiding light from one end of the capillary to the other. This property is very useful in the identification of capillaries in the bundling process, as we will describe in detail later. Second, the Germanium doping significantly increases the etching rate at the presence of hydrofluoric acid in comparison to the pure silica. Both the reaction well at the distal end and the fluid reservoir at the proximal end of the through hole can be fabricated by etching. This etching process can be carried out after the capillaries are grouped together to form an array. In addition to silica and Germanium doping, the capillary preform can also be assembled from two glass tubes, one inserted into the other, with the inner tube made of a type of glass that is easier to be etched away in comparison to that of the outer tube. The reaction well and the fluid reservoir can be produced the same way as described above.

A large number of individual capillaries are bundled together and solidified at the distal end by epoxy or heat welding. The solidified piece is cut, polished to a high degree of flatness to form a reaction plate. Then an array of reaction wells is fabricated on the plate surface and at the exit of each cavity using etching process described above. The proximal end of each capillary is inserted into a fluid reservoir containing a protein probe, respectively. The fluid reservoir can be a well in a standard microtiter plate. Multiple microtiter plates can be places inside a pressure chamber. Liquid probes are driven by pressure to fill the capillaries to form a liquid array.

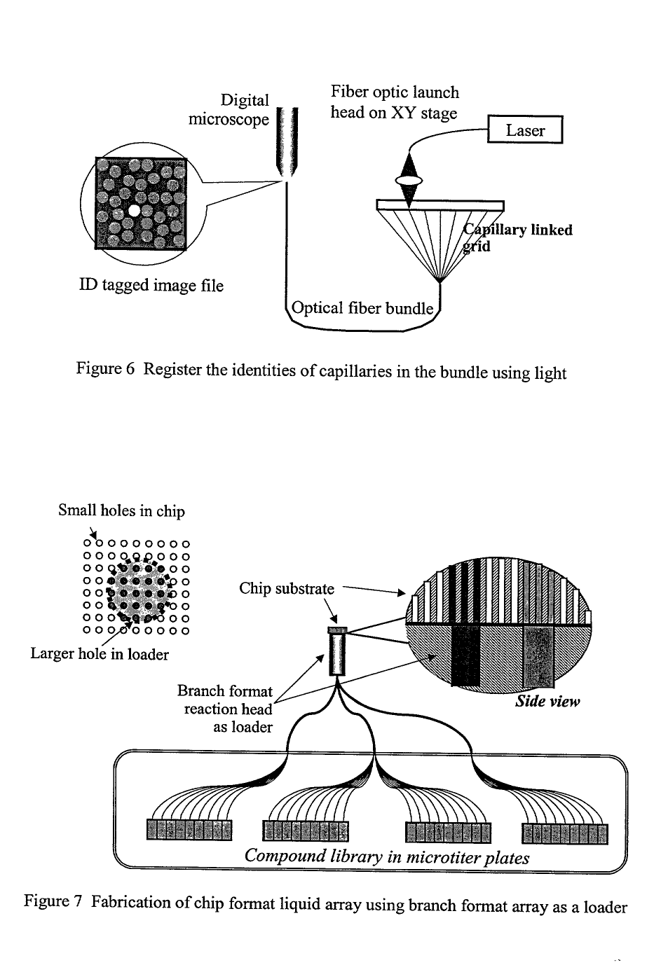

The spatial arrangement of the capillary array in the distal end can be either orderly or random. One issue rising from such a configuration is that the identities of each capillary, thus the probe, at distal end are lost in the bundling process because the capillaries are flexible and very small in diameter. The problem can be solved by optic fiber ID tagging. As described above, the capillaries used in the bundle are capable of conducting light. After the bundle is solidified at the distal end, light can be launched from the proximal end of each capillary, as shown in FIG. 6. A digital camera is used to observe and record the position of light exiting from the facet of the bundle at the distal end. After all capillary in the bundle are scanned, an ID tagged image file of the bundle can be built in computer, which registers the identities of each capillary.

Alternatively, after the bundle is solidified, a transparent fluid with higher refractive index than the capillary material is pumped into all capillaries. This creates a fluid waveguide in each capillary and enable their ID registration to be conducted by light as described above.

Bundle Format

The fluid array in bundle format can be fabricated direct from that in branch format. After individual liquid probes are pumped into capillaries. The proximal end of each capillary in the array can be taken out of the probe reservoir that it is inserted into and grouped together to form a capillary bundle that is bundled along its entire length. Liquid probes are stored within the cavities of capillaries and the stored volume is determined by the length of the capillary bundle and the inner diameter of the cavity. For example, a bundle of 1 m in length with a cavity diameter of 20 μm can store 0.3 μl probe liquid, sufficient for approximately 700 experiments. To increase the storage volume without lengthening the capillary, a section of the cavity can be enlarged by etching from the proximal end as described before.

The advantage of the bundle format is that the large pressure chamber that houses the microtiter plates in can be eliminated from the system that perform mixing and binding. This significantly reduces the cost and size of the instrument at the user end.

Chip Format

The liquid array in chip format can be fabricated in a number of ways. First, it can be made from the array in bundle format. A filled capillary bundle up to tens of meters can be frozen and then cut into shorter sections (chips). These chips can then thaw to produce liquid array in chip format. In the second method, many tube preforms are weld together to form a large honeycomb preform. This preform is extruded to a smaller diameter then welded with other similarly produced honeycombs to produce a new honeycomb preform with more through hole. This process can be repeated until the desired number of holes and pitch is reached. The honeycomb rod is then cut to produce chips contain a very large number of through holes. A chip containing millions of holes down to sub-micrometer in diameter can be produced in such a way. After the chip is made, the liquid probes can be loaded into the holes using the branch format liquid array in a system shown in FIG. 7. The holes in the branch format array can be precisely aligned to the holes in the chip.

In an alternative approach, each hole in the loader (the distal end of the branch format array) may cover several to hundreds of holes in the chip format, as shown in the enlarged section of FIG. 7. Because the cavity size in the chip is much smaller than that in the loader, the liquid is drawn into the chip by capillary force. Pressure may also be used to fill the through holes in the chip. In the chip format, therefore, a group of through holes contains the same probe. The reaction wells and reservoirs can be fabricated at the exit of each hole by etching as described before.

### APPLICATIONS

The protein array system has a wide number of applications, including, but not limited to, protein profiling and discovery, protein activity measurement, and identification of protein-protein and protein-small molecule interactions.

In one embodiment, the invention provides a method of determining protein expression profiles. One way of identifying diagnostic markers and novel disease targets is to compare the protein expression profile in normal vs. disease state. Proteins that show distinct expression pattern become candidates for diagnostic markers. They are also potential biotherapeutic agents or drug targets. One way to detect the repertories of protein expression is to use antibody array. Antibodies against known proteins can be arrayed and their interaction with proteins in a sample fluid can be determined using the array technology of this invention. Changes in protein expression profile in normal vs. disease state allow the identification of candidate proteins as biotherapeutic agents or as drug targets.

In one embodiment, the invention provides a method of discovering novel proteins of interest. It is possible to array antibodies against unknown proteins. For example, monoclonal antibodies can be raised against a mixture of antigens. The antigens in the mixture could be known or unknown proteins. There are many ways to create such a mixture. For example, a tissue sample of interest could be homogenized and further enriched through several chromatographic steps to enrich a fraction of total proteins. Antibodies raised against such a mixture may recognize majority or all of the proteins present in the mixture. Many of those proteins in the mixture could be unknown. The antibodies can be arrayed and used to detect the expression profile of proteins they recognize. Antibodies that recognize distinct pattern of expression profile in normal vs. disease state can be identified and their corresponding antigen could be novel proteins of interest.

In one embodiment, the invention provides a method of determining drug target. Pharmaceutical companies have many drug candidates that show therapeutic efficacy but could not be brought to the market. Some of those drug candidates may have undesirable side effect while others may have unknown mechanism of action. In those cases, the bottleneck has been that the protein targets for those candidate drugs are not known therefore the drug candidates could not be further optimized to have better ADME and toxicity profile. Furthermore, there are also drug candidates that may interact with multiple protein targets, some of which may cause side effect. In this case, it would be desirable to identify all those targets that cause undesirable side effect. In these instances, it is desirable to identify the drug target so that optimized drug can be developed. The drug candidates are allowed to interact with an array of repertories of proteins and potential protein targets can be identified through drug-protein interaction.

In one embodiment, the invention provides a method of determining protein-protein interactions. Traditionally, protein-protein interactions are measured through methods such as co-immunoprecipitation or yeast two-hybrid system. Co-immunoprecipitation method is often hampered by the lack of good antibody and by the amount of interacting proteins required. The yeast two-hybrid system is a labor-intensive and time-consuming process that requires further identification of the interacting proteins and it often generates high false-positive results. It is desirable to determine the interaction of a protein against known-repertories of protein arrays.

In one embodiment, the invention provides a method of identifying proteins that possess a desired biological activity. In many instances where a biological activity has been identified but the protein responsible for this activity is unknown. For example, a protein may be phosphorylated by an unknown kinase or a protein might be cleaved by an unknown protease. Currently, to identify a protein with such known activity, a very labor-intensive purification process based on an activity-based bioassay may be employed. It would be desirable to test the activity against known-repertories of protein arrays, thus allowing quick identification of the protein with the desired activity.

The liquid array system can be applied to other areas of life science as well. For example, the probes in the microtiter plates can also be DNA, chemical compounds, cells, or any material, substance or organisms that exist in solution. The target sample applied to the microwells can also be DNA, chemical compounds, cells, or any material, substance or organisms. Therefore, the liquid array system can also be adapted to DNA array, cell array, and chemical compound array.

What is claimed is:

1. A capillary bundle comprising a plurality of individual capillaries having proximal and distal ends, each of said capillaries having a channel extending from the proximal end to the distal end of the capillary and having a channel-facing wall, wherein each said channel contains a distinct probe in solution, said proximal ends of the individual capillaries are secured to one another in a solid mass such that the proximal ends of said capillaries are substantially coplanar in a static array in a facet of the solid mass, furthermore, each said proximal end comprises a reaction well fluidly connected to a channel, wherein the inner diameter of said reaction well is no less than 5 times larger than the inner diameter of said channel.

2. A capillary bundle according to claim 1, wherein said plurality of capillaries comprises light-conducting capillaries.

3. A capillary bundle according to claim 1, wherein the bundle has a print density of at least about 500 capillaries per cm² at the proximal end.

4. A capillary bundle according to claim 1, wherein the probe is selected from a group consisting of deoxyribonucleic acids (DNA), ribonucleic acids (RNA), synthetic oligonucleotides, antibodies, proteins, peptides, lectins, modified polysaccharides, synthetic composite macromolecules, functionalized nanostructures, synthetic polymers, modified/blocked nucleotides/nucleosides, modified/blocked amino acids, fluorophores, chromophores, ligands, chelates, haptens and drug compounds.
